(19) United States
(12) Patent Application Publication
(10) Pub. No.: US 2026/0335208 A1
(43) Pub. Date: Oct. 29, 2026

(54) COMBINATION COMPRISING A GLUCAMIDE AND A CO-EMULSIFIER

(71) Applicant: Clariant International Ltd, Muttenz (CH)

(72) Inventors: Tom FRICKE, Frankfurt am Main (DE); Ute BACK, Frankfurt am Main (DE)

(73) Assignee: Clariant International Ltd, Muttenz (CH)

(21) Appl. No.: 19/113,938

(22) PCT Filed: Sep. 28, 2023
(86) PCT No.: PCT/EP2023/076983

§ 371 Date: Mar. 21, 2025

(30) Foreign Application Priority Data
Oct. 6, 2022 (EP) 22200160.4

(51) Int. Cl. A61K 8/42; A61K 8/06; A61K 8/34; A61Q 19/00

(52) U.S. Cl. CPC A61K 8/42; A61K 8/06; A61K 8/34; A61K 8/345; A61Q 19/00; A61K 2800/10

(57) ABSTRACT

The present invention related to a combination comprising
(A) at least one compound of Formula (I),

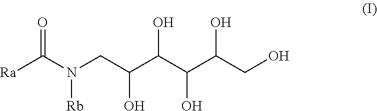

wherein
Ra is selected from saturated or unsaturated hydrocarbon chains having 5 to 23 carbon atoms, and mixtures thereof; or Ra—C═O is derived from high oleic sunflower oil, sunflower oil, olive oil, soybean oil, rapeseed oil or palm oil;
Rb is selected from C₁-C₄ alkyl;
(B) at least one co-emulsifier.

The present invention relates to a combination comprising a glucamide and a co-emulsifier, to the corresponding blends and cosmetic formulations as well as to the use of the combination as an emulsifier, solubilizer and/or surfactant.

Skin is constantly exposed to the environment and its changing conditions, such as dryness, humidity, sunshine, UV radiation, coldness, and pollution. Furthermore, under normal living conditions, skin is exposed to a microbial flora, including bacterial and fungal germs and cells. In addition, skin may be exposed to mechanical stress.

There is an ongoing need for cosmetic products, in particular skin care products, and ingredients therefor. In particular, there is a need for ingredients, such as emulsifiers, solubilizers and/or surfactants, that show good performance as well as are sustainable and environmentally friendly.

Consumers have generally become more conscious about the ingredients used in cosmetic products. Among others, they desire sustainable and environmentally friendly ingredients that are derived from renewable sources. Ideally, such ingredients have a low carbon footprint.

It has now been found that a combination comprising a glucamide and a co-emulsifier is useful as an emulsifier, solubilizer and/or surfactant.

The present invention relates to a combination comprising
(A) at least one compound of Formula (I),

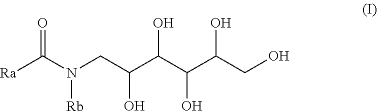

wherein
Ra is selected from saturated or unsaturated hydrocarbon chains having 5 to 23 carbon atoms, and mixtures thereof; or Ra—C═O is derived from high oleic sunflower oil, sunflower oil, olive oil, soybean oil, rapeseed oil or palm oil;
Rb is selected from C₁-C₄ alkyl;
(B) at least one co-emulsifier.

Advantageously, the combinations of the invention show good performance as an emulsifier, solubilizer and/or surfactant. They are sustainable and environmentally friendly. The combinations of the invention can be derived from renewable sources, such as sugars and natural oils, which may be locally sourced. Advantageously, the compound of Formula (I) has a low carbon footprint.

The combination of the invention comprises (A) at least one compound of Formula (I),

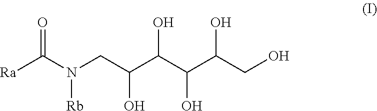

wherein
Ra is selected from saturated or unsaturated hydrocarbon chains having 5 to 23 carbon atoms, and mixtures thereof; or Ra—C═O is derived from high oleic sunflower oil, sunflower oil, olive oil, soybean oil, rapeseed oil or palm oil;
Rb is selected from C₁-C₄ alkyl.

A typical fatty acid composition of high oleic sunflower oil may be as follows:
3.8% palmitic acid (16)
3.0% stearic acid (18)
84.2% oleic acid (18:1, n-9)
7.6% linoleic acid (18:2, n-6)
0.1% linolenic acid (18:3alpha, n-3)

A typical fatty acid composition of sunflower oil may be as follows:
6.2% palmitic acid (16)
5% stearic acid (18)
19.9% oleic acid (18:1, n-9)
66.8% linoleic acid (18:2, n-6)

A typical fatty acid composition of olive oil may be as follows:
11.5% palmitic acid (16)
2.5% stearic acid (18)
0.5% icosanoic acid (20)
75.5% oleic acid (18:1, n-9)
7.5% linoleic acid (18:2, n-6)
1% linolenic acid (18:3alpha, n-3)

A typical fatty acid composition of soybean oil may be as follows:
10% palmitic acid (16)
4% stearic acid (18)
23% oleic acid (18:1, n-9)
51% linoleic acid (18:2, n-6)
7% (or <1%) linolenic acid (18:3alpha, n-3)

A typical fatty acid composition of rapeseed oil may be as follows:
4% palmitic acid (16)
1.5% stearic acid (18)
0.5% icosanoic acid (20)
63% oleic acid (18:1, n-9)
20% linoleic acid (18:2, n-6)
9% linolenic acid (18:3alpha, n-3)
1% gadoleic acid (20:1)

A typical fatty acid composition of palm oil may be as follows:
1% myristic acid
43.8% palmitic acid (16)
5% stearic acid (18)
0.5% icosanoic acid (20)
39% oleic acid (18:1, n-9)
10% linoleic acid (18:2, n-6)

Preferably, Ra in Formula (I) is selected from saturated or unsaturated hydrocarbon chains having 7 to 21 carbon atoms, and mixtures thereof; or Ra—C═O is derived from high oleic sunflower oil, sunflower oil, olive oil, soybean oil, rapeseed oil or palm oil; Rb in Formula (I) is selected from methyl and ethyl.

More preferably, Ra in Formula (I) is selected from saturated or unsaturated hydrocarbon chains having 11 to 21 carbon atoms, and mixtures thereof; or Ra—C═O is derived from high oleic sunflower oil, sunflower oil, olive oil, soybean oil or rapeseed oil; Rb in Formula (I) is selected from methyl and ethyl.

Even more preferably, Ra in Formula (I) is selected from saturated or unsaturated hydrocarbon chains having 15 to 17 carbon atoms, and mixtures thereof; or Ra—C═O is derived from high oleic sunflower oil, sunflower oil, olive oil, soybean oil or rapeseed oil; Rb in Formula (I) is methyl.

Particularly preferably, Ra in Formula (I) is selected from saturated or unsaturated hydrocarbon chains having 15 to 17 carbon atoms, and mixtures thereof; or Ra—C═O is derived from high oleic sunflower oil; Rb in Formula (I) is methyl.

Also preferably, Ra in Formula (I) is selected from saturated or unsaturated hydrocarbon chains having 7 to 17 carbon atoms, and mixtures thereof; or Ra—C═O is derived from high oleic sunflower oil, sunflower oil, olive oil, soybean oil or rapeseed oil; Rb in Formula (I) is selected from methyl and ethyl.

In a preferred embodiment, Ra in Formula (I) is selected from saturated or unsaturated hydrocarbon chains having 15 to 17 carbon atoms, and mixtures thereof; Rb in Formula (I) is methyl.

In a more preferred embodiment, Ra in Formula (I) is selected from saturated or unsaturated hydrocarbon chains having 15 or 17 carbon atoms, and mixtures thereof; Rb in Formula (I) is methyl.

In a particularly preferred embodiment, Ra in Formula (I) is selected from saturated hydrocarbon chains having 15 or 17 carbon atoms, and mixtures thereof; Rb in Formula (I) is methyl. The C16:C18 ratio may, for example, be from 0.5:9.5 to 7:3, preferably from 1:9 to 7:3, more preferably from 2:8 to 7:3, even more preferably from 3:7 to 7:3, particularly preferably from 4:6 to 6:4, for example 3:7, 4:6, 5:5, 6:4, or 7:3.

In a preferred embodiment, Ra—C═O in Formula (I) is derived from high oleic sunflower oil, sunflower oil, olive oil, soybean oil or rapeseed oil; Rb in Formula (I) is methyl.

In a particularly preferred embodiment, Ra—C═O in Formula (I) is derived from high oleic sunflower oil; Rb in Formula (I) is methyl. Such glucamide products are commercially available from Clariant as GlucoTain® GEM and GlucoTain® Sense.

In a preferred embodiment, the at least one compound of Formula (I) is used in the form of a composition comprising
(A) from 50 to 100 wt.-%, preferably from 60 to 95 wt.-%, more preferably from 65 to 90 wt.-%, particularly preferably from 70 to 80 wt.-%, of the at least one compound of Formula (I), based on the total weight of the composition;
(C) from 0 to 50 wt.-%, preferably from 5 to 40 wt.-%, more preferably from 10 to 35 wt.-%, particularly preferably from 20 to 30 wt.-%, of at least one alcohol, based on the total weight of the composition;
(D) from 0 to 10 wt.-%, preferably from 0 to 5 wt.-%, more preferably from 0 to 3 wt.-%, particularly preferably from 0 to 2 wt.-%, of water, based on the total weight of the composition.

In a preferred embodiment, the at least one compound of Formula (I) is used in the form of a composition comprising
(A) from 30 to 80 wt.-%, preferably from 40 to 70 wt.-%, more preferably from 45 to 60 wt.-%, particularly preferably from 50 to 54 wt.-%, of the at least one compound of Formula (I), based on the total weight of the composition;
(C) from 0 to 40 wt.-%, preferably from 5 to 40 wt.-%, more preferably from 20 to 40 wt.-%, particularly preferably from 36 to 40 wt.-%, of at least one alcohol, based on the total weight of the composition;
(D) from 0 to 60 wt.-%, preferably from 2 to 50 wt.-%, more preferably from 5 to 20 wt.-%, particularly preferably from 8 to 12 wt.-%, of water, based on the total weight of the composition.

Preferably, the alcohol (component C) is selected from ethanol, propanol, isopropanol, propylene glycol, 1,3-propanediol, butanol, isobutanol, butylene glycol, dipropylene glycol, glycerol and mixtures thereof. More preferably, the alcohol (component C) is selected from propylene glycol, 1,3-propanediol, glycerol, and mixtures thereof. Particularly preferably, the alcohol (component C) is propylene glycol or a mixture of propylene glycol and glycerol.

The combination of the invention comprises (B) at least one co-emulsifier.

Preferably, the at least one co-emulsifier (component B) is selected from fatty alcohols, fatty acids, glyceryl esters, sorbitan esters, sorbitol esters, polyglyceryl esters, sucrose esters, phosphates, fatty alcohol ethoxylates, lactylates, and mixtures thereof.

More preferably, the at least one co-emulsifier (component B) is selected from fatty alcohols, fatty acids, glyceryl esters, sorbitan esters, sorbitol esters, and mixtures thereof.

In a preferred embodiment, the at least one co-emulsifier (component B) is selected from fatty alcohols having 8 to 22 carbon atoms, fatty acids having 8 to 22 carbon atoms, esters of glycerol based on one or more C8-C22 fatty acids, esters of sorbitan based on one or more C8-C22 fatty acids, esters of sorbitol based on one or more C8-C22 fatty acids, esters of polyglycerol based on one or more C8-C22 fatty acids, esters of sucrose based on one or more C8-C22 fatty acids, esters of phosphoric acid based on one or more C8-C22 fatty alcohols or ethoxylated C8-C22 fatty alcohols, C8-C22 fatty alcohol ethoxylates, esters of lactic acid based on a C8-C22 fatty acid, and mixtures thereof.

In a more preferred embodiment, the at least one co-emulsifier (component B) is selected from fatty alcohols having 8 to 22 carbon atoms, fatty acids having 8 to 22 carbon atoms, esters of glycerol based on one or more C8-C22 fatty acids, esters of sorbitan based on one or more C8-C22 fatty acids, esters of sorbitol based on one or more C8-C22 fatty acids, and mixtures thereof.

In a preferred embodiment, the at least one co-emulsifier (component B) is selected from fatty alcohols having 12 to 22 carbon atoms, fatty acids having 12 to 22 carbon atoms, esters of glycerol based on one or more C12-C22 fatty acids, esters of sorbitan based on one or more C8-C22 fatty acids, esters of sorbitol based on one or more C12-C22 fatty acids, esters of polyglycerol based on one or more C8-C22 fatty acids, esters of sucrose based on one or more C12-C22 fatty acids, esters of phosphoric acid based on one or more C12-C22 fatty alcohols or ethoxylated C12-C22 fatty alcohols, C12-C22 fatty alcohol ethoxylates, esters of lactic acid based on a C12-C22 fatty acid, and mixtures thereof.

In a more preferred embodiment, the at least one co-emulsifier (component B) is selected from fatty alcohols having 12 to 22 carbon atoms, fatty acids having 12 to 22 carbon atoms, esters of glycerol based on one or more C12-C22 fatty acids, esters of sorbitan based on one or more C8-C22 fatty acids, esters of sorbitol based on one or more C12-C22 fatty acids, and mixtures thereof.

In a preferred embodiment, the at least one co-emulsifier (component B) is selected from fatty alcohols having 16 to 18 carbon atoms, fatty acids having 12 to 18 carbon atoms, esters of glycerol based on one or more C12-C18 fatty acids, esters of sorbitan based on one or more C8-C18 fatty acids, esters of sorbitol based on one or more C14-C18 fatty acids, esters of polyglycerol based on one or more C8-C18 fatty acids, esters of sucrose based on one or more C12-C18 fatty acids, esters of phosphoric acid based on one or more C12-C18 fatty alcohols or ethoxylated C12-C18 fatty alcohols, C12-C18 fatty alcohol ethoxylates, esters of lactic acid based on a C12-C18 fatty acid, and mixtures thereof.

In a more preferred embodiment, the at least one co-emulsifier (component B) is selected from fatty alcohols having 16 to 18 carbon atoms, fatty acids having 12 to 18 carbon atoms, esters of glycerol based on one or more C12-C18 fatty acids, esters of sorbitan based on one or more C8-C18 fatty acids, esters of sorbitol based on one or more C14-C18 fatty acids, and mixtures thereof.

The fatty alcohols can be saturated or unsaturated. The fatty acids can be saturated or unsaturated. The fatty acid residues in the above compounds can be saturated or unsaturated.

The fatty alcohols are, for example, selected from cetearyl alcohol, cetyl alcohol, stearyl alcohol, behenyl alcohol, lauryl alcohol, myristyl alcohol, and mixtures thereof.

The fatty acids are, for example, selected from stearic acid, palmitic acid, behenic acid, lauric acid, myristic acid, oleic acid, ricinoleic acid, and mixtures thereof.

The glyceryl esters are, for example, selected from glyceryl stearate, glyceryl stearate citrate, glyceryl oleate, glyceryl laurate, glyceryl myristate, glyceryl palmitate, glyceryl cocoate, glyceryl ricinoleate, and mixtures thereof.

The sorbitan esters are, for example, selected from sorbitan stearate, sorbitan isostearate, sorbitan olivate, sorbitan oleate, sorbitan sesquioleate, sorbitan caprylate, sorbitan laurate, sorbitan palmitate, and mixtures thereof. Sorbitan caprylate is commercially available from Clariant (Velsan® SC).

The sorbitol esters are, for example, sorbitol esters derived from sunflower oil or high oleic sunflower oil. Sunflower Seed Oil Sorbitol Esters is commercially available from Clariant (Plantasens® Emulsifier SFO) Preferably, the number of glyceryl units in the polyglyceryl esters is from 2 to 20, more preferably from 2 to 4, even more preferably 2 or 3, particularly preferably 2. The polyglyceryl esters are, for example, selected from polyglyceryl-2 stearate, polyglyceryl-3 stearate, polyglyceryl-4 stearate, polyglyceryl-2 sesquistearate, polyglyceryl-2 sesquiisostearate, polyglyceryl-2 caprate, polyglyceryl-3 caprate, polyglyceryl-4 caprate, polyglyceryl-2 caprylate, polyglyceryl-3 caprylate, polyglyceryl-4 caprylate, polyglyceryl-2 laurate, polyglyceryl-3 laurate, polyglyceryl-4 laurate, polyglyceryl-2 myristate, polyglyceryl-3 myristate, polyglyceryl-4 myristate, polyglyceryl-2 palmitate, polyglyceryl-3 palmitate, polyglyceryl-4 palmitate, polyglyceryl-2 oleate, polyglyceryl-3 oleate, polyglyceryl-4 oleate, polyglyceryl-2 cocoate, polyglyceryl-3 cocoate, polyglyceryl-4 cocoate, polyglyceryl-2 ricinoleate, polyglyceryl-3 ricinoleate, polyglyceryl-4 ricinoleate, and mixtures thereof. Polyglyceryl-2 stearate is, e.g., commercially available from Clariant (Plantasens® Emulsifier DGDS). Polyglyceryl-2 sesquiisostearate is, e.g. commercially available from Clariant (Plantasens® Emulsifier DGI).

The sucrose esters are, for example, selected from sucrose cocoate, sucrose dilaurate, sucrose stearate, sucrose distearate, sucrose polystearate, sucrose laurate, sucrose myristate, sucrose oleate, sucrose palmitate, sucrose ricinoleate, and mixtures thereof.

The phosphates are, for example, selected from cetyl phosphate, trilaureth-4 phosphate, triceteareth-4 phosphate, and mixtures thereof.

Preferably, the number of ethyleneoxy (—CH₂CH₂O—) units in the fatty alcohol ethoxylates is from 1 to 30, more preferably from 5 to 25, particularly preferably from 5 to 20. The fatty alcohol ethoxylates are, for example, selected from ethoxylated stearyl alcohols, ethoxylated isostearyl alcohols, ethoxylated cetyl alcohols, ethoxylated isocetyl alcohols, ethoxylated oleyl alcohols, ethoxylated lauryl alcohols, ethoxylated isolauryl alcohols, ethoxylated cetylstearyl alcohols, and mixtures thereof. The fatty alcohol ethoxylates are, for example, selected from polyethylene glycol (13) stearyl ether, polyethylene glycol (14) stearyl ether, polyethylene glycol (15) stearyl ether, polyethylene glycol (16) stearyl ether, polyethylene glycol (17) stearyl ether, polyethylene glycol (18) stearyl ether, polyethylene glycol (19) stearyl ether, polyethylene glycol (20) stearyl ether, polyethylene glycol (12) isostearyl ether, polyethylene glycol (13) isostearyl ether, polyethylene glycol (14) isostearyl ether, polyethylene glycol (15) isostearyl ether, polyethylene glycol (16) isostearyl ether, polyethylene glycol (17) isostearyl ether, polyethylene glycol (18) isostearyl ether, polyethylene glycol (19) isostearyl ether, polyethylene glycol (20) isostearyl ether, polyethylene glycol (13) cetyl ether, polyethylene glycol (14) cetyl ether, polyethylene glycol (15) cetyl ether, polyethylene glycol (16) cetyl ether, polyethylene glycol (17) cetyl ether, polyethylene glycol (18) cetyl ether, polyethylene glycol (19) cetyl ether, polyethylene glycol (20) cetyl ether, polyethylene glycol (13) isocetyl ether, polyethylene glycol (14) isocetyl ether, polyethylene glycol (15) isocetyl ether, polyethylene glycol (16) isocetyl ether, polyethylene glycol (17) isocetyl ether, polyethylene glycol (18) isocetyl ether, polyethylene glycol (19) isocetyl ether, polyethylene glycol (20) isocetyl ether, polyethylene glycol (12) oleyl ether, polyethylene glycol (13) oleyl ether, polyethylene glycol (14) oleyl ether, polyethylene glycol (15) oleyl ether, polyethylene glycol (12) lauryl ether, polyethylene glycol (12) isolauryl ether, polyethylene glycol (13) cetylstearyl ether, polyethylene glycol (14) cetylstearyl ether, polyethylene glycol (15) cetylstearyl ether, polyethylene glycol (16) cetylstearyl ether, polyethylene glycol (17) cetylstearyl ether, polyethylene glycol (18) cetylstearyl ether, polyethylene glycol (19) cetylstearyl ether, ceteareth-20, and mixtures thereof.

The lactylates are, for example, selected from sodium stearoyl lactylate, sodium lauroyl lactylate, and mixtures thereof.

In a particularly preferred embodiment, the at least one co-emulsifier (component B) is selected from cetearyl alcohol, cetyl alcohol, stearyl alcohol, stearic acid, palmitic acid, glyceryl stearate, sorbitan stearate, sorbitol esters derived from sunflower oil or high oleic sunflower oil, and mixtures thereof.

In a preferred embodiment, the weight ratio of (A) the at least one compound of Formula (I) to (B) the at least one co-emulsifier is from 5:1 to 1:30, preferably from 2:1 to 1:20, more preferably from 1:1 to 1:15, even more preferably from 1:2 to 1:10, particularly preferably from 1:3 to 1:6.

The combination of the invention can, for example, be used in a cosmetic formulation. The components of the combination of the invention can, for example, be added to a cosmetic formulation separately or in the form of a blend.

In one embodiment, the combination is in the form of a blend.

The present invention also relates to a blend comprising
(A) from 2 to 50 wt.-%, preferably from 5 to 40 wt.-%, more preferably from 5 to 35 wt.-%, particularly preferably from 5 to 25 wt.-%, of at least one compound of Formula (I) as described herein, based on the total weight of the blend;
(B) from 40 to 95 wt.-%, preferably from 50 to 90 wt.-%, more preferably from 60 to 90 wt.-%, particularly preferably from 70 to 90 wt.-%, of at least one co-emulsifier as described herein, based on the total weight of the blend;
(C) from 0 to 20 wt.-%, preferably from 1 to 15 wt.-%, more preferably from 1 to 12 wt.-%, particularly preferably from 2 to 10 wt.-%, of at least one alcohol, based on the total weight of the blend;
(D) from 0 to 5 wt.-%, preferably from 0 to 3 wt.-%, more preferably from 0 to 2 wt.-%, particularly preferably from 0 to 1 wt.-%, of water, based on the total weight of the blend.

Preferably, the alcohol (component C) is selected from ethanol, propanol, isopropanol, propylene glycol, 1,3-propanediol, butanol, isobutanol, butylene glycol, dipropylene glycol, glycerol, and mixtures thereof. More preferably, the alcohol (component C) is selected from propylene glycol, 1,3-propanediol, glycerol, and mixtures thereof. Particularly preferably, the alcohol (component C) is propylene glycol or a mixture of propylene glycol and glycerol.

The blend of the invention can be prepared by methods known in the art, for example by mixing its components. The blend of the invention can, for example, be liquid or solid. In one embodiment, the blend is in the form of flakes or pellets. Flakes or pellets can, for example, be prepared by mixing its components, heating the resulting mixture to obtain a melt, bringing the melt in contact with a cold surface in a manner known in the art to obtain flakes or pellets.

The combination of the invention, or the composition as described herein, or the blend of the invention can, for example be used as an emulsifier, solubilizer and/or surfactant. In a preferred embodiment, the combination of the invention, or the composition as described herein, or the blend of the invention is used as an emulsifier. Preferably, the combination of the invention, or the composition as described herein, or the blend of the invention is used in a cosmetic formulation, more preferably a skin care formulation, particularly preferably a leave-on skin care formulation.

The present invention also relates to the use of a combination of the invention, or a composition as described herein, or a blend of the invention as an emulsifier, solubilizer and/or surfactant. In a preferred embodiment, the present invention relates to the use of a combination of the invention, or a composition as described herein, or a blend of the invention as an emulsifier. In a particularly preferred embodiment, the present invention relates to the use of a combination of the invention, or a composition as described herein, or a blend of the invention as an emulsifier in a cosmetic formulation, preferably a skin care formulation, particularly preferably a leave-on skin care formulation.

The present invention also relates to a formulation comprising
(A) from 0.5 to 5 wt.-%, preferably from 1 to 4 wt.-%, more preferably from 2 to 4 wt.-%, particularly preferably from 2 to 3 wt.-%, of at least one compound of Formula (I) as described herein, based on the total weight of the formulation;
(B) from 2 to 20 wt.-%, preferably from 3 to 16 wt.-%, more preferably from 6 to 16 wt.-%, particularly preferably from 6 to 12 wt.-%, of at least one co-emulsifier as described herein, based on the total weight of the formulation.

In a preferred embodiment, the formulation of the present invention is a cosmetic formulation, preferably a skin care formulation, particularly preferably a leave-on skin care formulation.

The formulation of the present invention may, for example, be a skin care, hair care and/or scalp care formulation. In a particularly preferred embodiment, the formulation is a skin care formulation. In another preferred embodiment, the formulation is a hair care formulation. The formulation of the present invention may, for example, be applied to skin, hair and/or scalp. Typically, the formulation is applied topically to skin, hair and/or scalp. In a particularly preferred embodiment, the formulation is applied to skin. Typically, the formulation is applied topically to skin. The formulation of the present invention may, for example, be a leave-on formulation or a rinse-off formulation. In a particularly preferred embodiment, the formulation is a leave-on skin care formulation. In another preferred embodiment, the formulation is a leave-on hair care formulation.

In another preferred embodiment, the formulation of the present invention may be a pharmaceutical formulation. In another preferred embodiment, the formulation of the present invention may be a dermatological formulation.

In a preferred embodiment, the formulation is a cosmetic formulation selected from the group consisting of skin care oil, skin care cream, skin care lotion, ointment, skin conditioner, face spray, body spray, hand oil, shower bath, hair conditioner, shaving gel, shampoo, body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, lip stick, lip gloss, body oil, shower milk, illuminator, lip crayon, hair spray, combing cream, and sunblock.

In a preferred embodiment, the formulation is a cosmetic formulation selected from the group consisting of day cream, anti-aging cream, body milk, body lotion, body mousse, face serum, eye cream, sunscreen lotion, sun cream, face cream, after-shave lotion, skin-whitening gel, self-tanning cream, anti-acne gel, foundation, primer, concealer, blemish balm (bb) cream, night cream, eye brow gel, hand sanitizer, skin conditioner, deodorant, antiperspirant, baby cream, insect repellent, hand cream, sunscreen gel, foot cream, cellulite treatment, nail cuticle cream, lip balm, body mist, eau de toilette, lubricating gel, moisturizer, serum, toner, aqua sorbet, cream gel, body oil, illuminator, and sunblock.

In a particularly preferred embodiment, the formulation is a cosmetic formulation selected from the group consisting of day cream, anti-aging cream, body milk, body lotion, face serum, eye cream, sunscreen lotion, face cream, night cream, baby cream, hand cream, foot cream, nail cuticle cream, lip balm, moisturizer, body oil, and sunblock.

In at least one embodiment, the formulation comprises a solvent. In at least one embodiment, the formulation comprises a solvent, wherein the solvent comprises water and/or alcohol. Solvent is useful for providing the compounds used in present invention in liquid form. In at least one embodiment, the solvent is cosmetically acceptable. In at least one embodiment, the formulation comprises at least 10 wt.-%, preferably at least 20 wt.-%, more preferably at least 30 wt.-%, even more preferably at least 50 wt.-% water. Water is useful for economic reasons but also because it is cosmetically acceptable. Optionally, the formulation comprises water-miscible or water-soluble solvents, such as lower alkyl alcohols. In at least one embodiment, the formulation comprises C₁-C₅ alkyl monohydric alcohols, preferably C2-C3 alkyl monohydric alcohols. The alcohols which may be present are in particular lower monohydric or polyhydric alcohols having 1 to 4 carbon atoms customarily used for cosmetic purposes, such as preferably ethanol and isopropanol.

In a preferred embodiment, the formulation comprises a solvent selected from the group consisting of water, glycols, ethanol, and mixtures thereof. In a preferred embodiment, the formulation comprises water. In one embodiment, the formulation is an aqueous solution.

In a preferred embodiment, the formulation comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

In at least one embodiment, the formulation comprises additives common in cosmetology, pharmacy, and dermatology, which are hereinafter called auxiliaries. In at least one embodiment, the auxiliary is selected from the group consisting of oily substances, cationic polymers, film formers, superfatting agents, stabilizers, active biogenic substances, glycerol, preservatives, pearlizing agents, dyes and fragrances, solvents, opacifiers, functional acids, and also protein derivatives such as gelatin, collagen hydrolysates, natural or synthetic-based polypeptides, egg yolk, lecithin, lanolin and lanolin derivatives, fatty alcohols, silicones, deodorants, substances with a keratolytic and keratoplastic action, enzymes, and/or carriers/solvents.

In at least one embodiment, the formulation comprises water soluble vitamins and their derivatives, water soluble amino acids and their salts and/or derivatives, viscosity modifiers, dyes, nonvolatile solvents or diluents (water soluble and insoluble), pearlescent aids, thickeners, foam boosters, surfactants or co-surfactants, pediculocides, pH adjusting agents, perfumes, preservatives, chelants, proteins, skin active agents, sunscreens, UV absorbers, vitamins, niacinamide, caffeine, minoxidil, and combinations thereof. In at least one embodiment, the formulation comprises from 0 wt.-% to 5 wt.-% vitamins and amino acids, by total weight of the formulation.

In at least one embodiment, the formulation comprises an oily substance, which is any fatty substance which is liquid at room temperature (25° C.). In at least one embodiment, the formulation comprises oily substance selected from the group consisting of silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C₆-C₁₃) fatty acids with linear (C₆-C₂₀) fatty alcohols; esters of branched (C₆-C₁₃) carboxylic acids with linear (C₆-C₂₀) fatty alcohols, esters of linear (C₆-C₁₈) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C₆-C10) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds.

In at least one embodiment, the formulation comprises a cationic polymer. Suitable cationic polymers include those known under the INCI designation “Polyquaternium”, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and also Polyquaternium 37 & mineral oil & PPG trideceth (Salcare SC95), PVP dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

In at least one embodiment, the formulation comprises a superfatting agent. As superfatting agents it is possible to use substances such as, for example, polyethoxylated lanolin derivatives, lecithin derivatives, polyol fatty acid esters, monoglycerides, or fatty acid alkanol amides, the latter serving simultaneously as foam stabilizers. Moisturizers available include for example isopropyl palmitate, glycerol and/or sorbitol.

In at least one embodiment, the formulation comprises a stabilizer. As stabilizer it is possible to use metal salts of fatty acids, such as magnesium, aluminum and/or zinc stearate, for example.

In at least one embodiment, the formulation comprises a care additive. The formulations can be blended with conventional ceramides, pseudoceramides, cholesterol, cholesterol fatty acid esters, cerebrosides, phospholipids, panthenol and similar substances as a care additive.

In at least one embodiment, the formulation comprises a preservative or preservative system. Examples of suitable preservatives include benzyl alcohol, piroctone olamine, phenoxyethanol, parabens, pentanediol, benzoic acid/sodium benzoate, sorbic acid/potassium sorbate, caprylhydroxamic acid, or combinations thereof. Examples of suitable preservation boosting ingredients include anisic acid, lactic acid, sorbitan caprylate, ethylhexylglycerin, methylheptylglycerin, caprylyl glycol, caprylyl glyceryl ether, octanediol, Capryloyl/Caproyl Anhydro Methyl Glucamide (Velsan® Flex), or combinations thereof. In at least one embodiment, the formulation comprises from 0.01 to 5 wt.-%, particularly preferably from 0.05 to 1 wt.-% of at least one preservative.

In at least one embodiment, the formulation comprises a preservative selected from the group consisting of cetyltrimethyl ammonium chloride, cetylpyridinium chloride, benzethonium chloride, diisobutylethoxyethyldimethyl benzylammonium chloride, sodium N-lauryl sarcosinate, sodium N-palmethylsarcosinate, lauroylsarcosine, N-myristoylglycine, potassium-N-laurylsarcosine, trimethylammonium chloride, sodium aluminium chlorohydroxylactate, triethylcitrate, tricetylmethylammonium chloride, 2,4,4′-trichloro-2′-hydroxydiphenylether (Triclosan), phenoxyethanol, 1,5-pentanediol, 1,6-hexanediol, 3,4,4′-trichlorocarbanilide (Triclocarban), diaminoalkylamide, L-lysine hexadecylamide, heavy metal citrate salts, salicylate, piroctone, zinc salts, pyrithione and its heavy metal salts, zinc pyrithione, zinc phenol sulfate, farnesol, ketoconazol, oxiconazol, bifonazole, butoconazole, cloconazole, clotrimazole, econazole, enilconazole, fenticonazole, isoconazole, miconazole, sulconazole, tioconazole, fluconazole, itraconazole, terconazole, naftifine, terbinafine, selenium disulfide, methylchloroisothiazolinone, methylisothiazolinone, methyldibromo glutaronitrile, piroctone olamine (Octopirox®), AgCI, chloroxylenol, sodium salts of diethylhexylsulfosuccinate, sodium benzoate, phenoxyethanol, benzyl alcohol, phenoxyisopropanol, paraben, such as butyl-, ethyl-, methyl- and propylparaben, and their salts, pentanediol, 1,2-octanediol, ethylhexylglycerin, benzyl alcohol, sorbic acid, benzoic acid, lactic acid, imidazolidinyl urea, diazolidinyl urea, dimethylol dimethyl hydantoin (DMDMH), sodium salts of hydroxymethyl glycinate, hydroxyethylglycine of sorbic acid, and combinations thereof. In at least one embodiment, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin, and combinations thereof. In at least one embodiment, the formulation is substantially free of parabens.

In at least one embodiment, the formulation comprises an anti-fungal substance. In at least one embodiment, the anti-fungal substance is selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, zinc pyrithione, piroctone olamine (octopirox), and combinations thereof. In at least one embodiment, the formulation comprises a total amount of anti-fungal substance in the formulation of from 0.1 wt.-% to 1 wt.-%. In at least one embodiment, the formulation comprises pyridinethione anti-dandruff particulates. For example, 1-hydroxy-2-pyridinethione salts are highly preferred particulate anti-dandruff agents. The concentration of pyridinethione antidandruff particulate may range from 0.1 wt.-% to 4 wt.-%, by total weight of the formulation, preferably from 0.1 wt.-% to 3 wt.-%, more preferably from 0.3 wt.-% to 2 wt.-%. Preferred pyridinethione salts include those formed from heavy metals such as zinc, tin, cadmium, magnesium, aluminum or zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form. Salts formed from other cations, such as sodium, may also be suitable.

Functional acids are acidic substances used to impart a clinical functionality to the skin or hair upon application. Suitable functional acids include alpha-hydroxy acids, beta-hydroxy acids, lactic acid, retinoic acid, and similar substances.

In at least one embodiment, the formulation comprises an astringent. In at least one embodiment, the astringent is selected from the group consisting of magnesium oxide, aluminum oxide, titanium dioxide, zirconium dioxide, zinc oxide, oxide hydrates, aluminum oxide hydrate (boehmite) and hydroxide, chlorohydrates of calcium, magnesium, aluminum, titanium, zirconium or zinc. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% astringent.

In at least one embodiment, the formulation comprises a deodorizing agent. In at least one embodiment, the deodorizing agent is selected from the group consisting of allantoin, bisabolol, and combinations thereof. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% deodorizing agent.

In at least one embodiment, the formulation comprises a sun protection agent and/or UV filter. Suitable sun protection agents and UV filters are disclosed in WO-2013/017262A1, from page 32, line 11 to the end of page 33. In at least one embodiment, the sun protection agent and/or UV filter is selected from the group consisting of 4-amino benzoic acid, 3-(4′-trimethylammonium)-benzylide-boran-2-one-methylsulfate, camphor benzalkonium methosulfate, 3,3,5-trimethyl-cyclohexylsalicylate, 2-hydroxy-4-methoxybenzophenone, 2-phenylbenzimidazole-5-sulfonic acid and potassium-, sodium- und triethanolamine salts thereof, 3,3′-(1,4-phenylene dimethine)-bis-(7,7-dimethyl-2-oxobicyclo[2.2.1]-heptane-1-methane sulfonic acid) and its salts, 1-(4-tert.-butylphenyl)-3-(4-methoxyphenyl)propan-1,3-dion, 3-(4′-sulfo)-benzylidene-bornane-2-one its salts, 2-cyan-3,3-diphenyl-acrylic acid-(2-ethylhexylester), polymers of N-[2(and 4)-(2-oxoborn-3-ylidenmethyl)benzyl]-acrylamide, 4-methoxy-cinnamic acid-2-ethyl-hexylester, ethoxylated ethyl-4-amino-benzoate, 4-methoxy-cinnamic acid-isoamylester, 2,4,6-tris-[p-(2-ethylhexyloxycarbonyl)anilino]-1,3,5-triazine, 2-(2H-benzotriazole-2-yl)-4-methyl-6-(2-methyl-3-(1,3,3,3-tetramethyl-1-(trimethylsilyloxy)-disiloxanyl)-propyl)phenol, 4,4′-[(6-[4-((1,1-dimethylethyl)-amino-carbonyl)phenylamino]-1,3,5-triazin-2,4-yl)diimino]bis-(benzoic acid-2-ethylhexylester), 3-benzophenone, 4-benzophenone (acid), 3(4′-methylbenzyliden)-D,L-camphor, 3-benzylidene-camphor, salicylic acid-2-ethylhexylester, 4-dimethyl aminobenzic acid-2-ethylhexylester, hydroxy-4-methoxy-benzophenone-5 sulfonic acid and the sodium salt thereof, 4-isopropyl benzylsalicylate, N,N,N-trimethyl-4-(2-oxoborn-3-ylidenemethyl) anilium methyl sulfate, homosalate (INN), oxybenzone (INN), 2-phenylbenzimidazole-5-sulfonic acid and its sodium, potassium, and triethanolamine salts, octylmethoxy cinnamic acid, isopentyl-4-methoxy cinnamic acid, isoamyl-p-methoxy cinnamic acid, 2,4,6-trianilino-(p-carbo-2′-ethylhexyl-1′-oxy)-1,3,5-triazine (octyl triazone) phenol, 2,2(2H-benzotriazol-2-yl)-4-methyl-6-(2-methyl-3-(1,3,3,3-tetramethyl-1-(trimethylsilyl)oxy)-disiloxanyl)propyl (drometrizole trisiloxane) benzic acid, 4,4-((6-(((1,1-dimethylethyl)amino)carbonyl)phenyl)amino)-1,3,5-triazine-2,4-diyl)diimino)bis,bis(2-ethylhexyl)ester) benzoic acid, 4,4-((6-(((1,1-dimethylethyl)amino)-carbonyl)phenyl)amino)-1,3,5-triazine-2,4-diyl)diimino)bis,bis(2-ethylhexyl)ester), 3-(4′-methylbenzylidene)-D,L-camphor (4-methylbenzylidene camphor), benzylidene-camphor-sulfonic acid, octocrylene, polyacrylamidomethyl-benzylidene-camphor, 2-ethylhexyl salicylate (octyl salicylate), 4-dimethyl-aminobenzoeacidethyl-2-hexylester (octyl dimethyl PABA), PEG-25 PABA, 2 hydroxy-4-methoxybenzo-phenone-5-sulfonic acid (5-benzophenone) and the sodium salt thereof, 2,2′-methylene-bis-6-(2H-benzotriazol-2-yl)-4-(tetramethyl-butyl)-1,1,3,3-phenol, the sodium salt of 2-2′-bis-(1,4-phenylene)1H-benzimidazole-4,6-disulfonic acid, (1,3,5)-triazine-2,4-bis((4-(2-ethyl-hexyloxy)-2-hydroxy)-phenyl)-6-(4-methoxyphenyl), 2-ethylhexyl-2-cyano-3,3-diphenyl-2-propenoate, glyceryl octanoate, di-p-methoxy cinnamic acid, p-amino-benzoic acid and its ester, 4-tert-butyl-4′-methoxydibenzoylmethane, 4-(2-p-glucopyranoxy)propoxy-2-hydroxybenzophenone, octyl salicylate, methyl-2,5-diisopropyl cinnamic acid, cinoxate, dihydroxy-dimethoxybenzophenone, disodium salts of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl-dimethoxybenzyliden-dioxoimidazolidinpropionate, methylene-bis-benztriazolyl tetramethylbutylphenol, phenyldibenzimidazoltetrasulfonate, bis-ethylhexyloxyphenol-methoxyphenol-triazine, tetrahydroxybenzophenone, terephthalylidendicamphor-sulfonic acid, 2,4,6-tris[4,2-ethylhexyloxycarbonyl)anilino]-1,3,5-triazine, methyl-bis(trimethylsiloxy)silyl-isopentyl trimethoxy cinnamic acid, amyl-p-dimethylaminobenzoate, amyl-p-dimethylamino benzoate, 2-ethylhexyl-p-dimethylaminobenzoate, isopropyl-p-methoxy cinnamic acid/diisopropyl cinnamic acid ester, 2-ethylhexyl-p-methoxy cinnamic acid, 2-hydroxy-4-methoxy benzophenone, 2-hydroxy-4-methoxybenzophenone-5-sulfonic acid and the trihydrate, 2-hydroxy-4-methoxybenzophenone-5-sulfonate sodium salt, phenyl-benzimidazole sulfonic acid, and combinations thereof. In at least one embodiment, the formulation comprises from 0.001 wt.-% to 10 wt.-%, preferably from 0.05 wt.-% to 5 wt.-%, even more preferably from 0.1 wt.-% to 3 wt.-%, most preferably from 0.05 wt.-% to 1 wt.-% sun protection agent and/or UV filter. In at least one embodiment, the formulation comprises a photoprotective substance in an amount of from 0.01 to 10 wt.-%, or from 0.1 to 5 wt.-%, more preferably from 0.2 to 2 wt.-%. Suitable photoprotective substances include, in particular, all of the photoprotective substances specified in EP1084696A1, which is incorporated herein by reference. In a preferred embodiment, the photoprotective substance is selected from the group consisting of 2-ethylhexyl 4-methoxycinnamate, methyl methoxycinnamate, 2-hydroxy-4-methoxybenzophenone-5-sulfonic acid, polyethoxylated p-aminobenzoates, and combinations thereof.

In at least one embodiment, the formulation comprises an anti-oxidant. In at least one embodiment, the anti-oxidant is selected from the group consisting of amino acids, peptides, sugars, imidazoles, carotinoids, carotenes, chlorogenic acid, lipoic acid, thiols, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, thiol glyceryl esters, dilaurylthiodipropionate, distearylthiodipropionate, thiodipropionic acid, metal chelators, hydroxy acids, fatty acids, folic acids, vitamin C, tocopherol, vitamin A, stilbenes, derivatives and combinations thereof. In at least one embodiment, the anti-oxidant is selected from the group consisting of glycine, histidine, tyrosine, tryptophan, urocaninic acid, D,L-carnosine, D-carnosine, L-carnosine, beta-carotene, alpha-carotene, lycopene, dihydrolipoic acid, aurothioglucose, propylthiouracil, thioredoxine, glutathione, cysteine, cystine, cystamine, buthioninsulfoximine, homocysteinsulfoximine, buthioninsulfone, penta-, hexa-, heptathioninsulfoximine, hydroxyfatty acids, palmitic acid, phytinic acid, lactoferrin, citric acid, lactic acid, malic acid, humic acid, bile acid, bilirubin, biliverdin, EDTA, EGTA, linoleic acid, linolenic acid, oleic acid, butylhydroxyanisol, trihydroxybutyrophenone, ubichinon, ubichinol, ascorbylpalmitate, Mg-ascorbylphosphate, ascorbylacetate, vitamin E acetate, vitamin A palmitate, carnosine, mannose, ZnO, ZnSO4, selenium methionine, stilbenes, superoxide dismutase, and combinations thereof. In at least one embodiment, the antioxidant is selected from the group consisting of vitamin A, vitamin A derivatives, vitamin E, vitamin E derivatives, and combinations thereof. In at least one embodiment, the formulation comprises from 0.001 wt.-% to 10 wt.-%, preferably from 0.05 wt.-% to 5 wt.-%, particularly preferably from 0.1 wt.-% to 3 wt.-%, also particularly preferably from 0.05 wt.-% to 1 wt.-% antioxidant.

In at least one embodiment, the formulation comprises a dye or pigment. In at least one embodiment, the formulation comprises at least one pigment. Suitable dyes and pigments are disclosed in WO2013/017262A1 in the table spanning pages 36 to 43. These may be colored pigments which impart color effects to the product mass or to hair, or they may be luster effect pigments which impart luster effects to the product mass or to hair. The color or luster effects on hair are preferably temporary, i.e. they last until the next hair wash and can be removed again by washing the hair with customary shampoos. In at least one embodiment, the formulation comprises a total amount of from 0.01 wt.-% to 25 wt.-%, preferably from 5 wt.-% to 15 wt.-% pigment. In at least one embodiment, the particle size of the pigment is from 1 micron to 200 micron, preferably from 3 micron to 150 micron, more preferably from 10 micron to 100 micron. The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. In at least one embodiment, the inorganic pigment is selected from the group consisting of chalk, ochre, umber, green earth, burnt sienna, graphite, and combinations thereof. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, where preferably at least one pigment is a colored, nonwhite pigment. In at least one embodiment, the pigment is selected from the group consisting of metal oxides, hydroxides and oxide hydrates, mixed phase pigments, sulfur-containing silicates, metal sulfides, complex metal cyanides, metal sulfates, chromates and molybdates, and the metals themselves (bronze pigments), and combinations thereof. In at least one embodiment, the pigment is selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), and combinations thereof. In at least one embodiment, the pigment is selected from the group consisting of pearlescent and colored pigments based on mica which are coated with a metal oxide or a metal oxychloride, such as titanium dioxide or bismuth oxychloride, and optionally further color-imparting substances, such as iron oxides, Prussian blue, ultramarine, carmine etc. and where the color can be determined by varying the layer thickness. Such pigments are sold, for example, under the trade names Rona®, Colorona®, Dichrona® and Timiron® by Merck. In at least one embodiment, the pigment is selected from the group consisting of organic pigments such as sepia, gamboge, bone charcoal, Cassel brown, indigo, chlorophyll and other plant pigments. In at least one embodiment, the pigment is selected from the group consisting of synthetic organic pigments such as azo pigments, anthraquinoids, indigoids, dioxazine, quinacridone, phthalocyanine, isoindolinone, perylene and perinone, metal complex, alkali blue and diketopyrrolopyrrole pigments.

In at least one embodiment, the formulation comprises from 0.01 wt.-% to 10 wt.-%, preferably from 0.05 wt.-% to 5 wt.-%, of at least one particulate substance. Suitable substances are, for example, substances which are solid at room temperature (25° C.) and are in the form of particles. In at least one embodiment, the particulate substance is selected from the group consisting of silica, silicates, aluminates, clay earths, mica, insoluble salts, in particular insoluble inorganic metal salts, metal oxides, e.g. titanium dioxide, minerals and insoluble polymer particles are suitable. The particles may be present in the formulation in undissolved, preferably stably dispersed form, and, following application to the keratin substrate and evaporation of the solvent, can deposit on the substrate in solid form. A stable dispersion can be achieved by providing the formulation with a yield point which is large enough to prevent the solid particles from sinking. An adequate yield point can be established using suitable gel formers in a suitable amount. In at least one embodiment, the particulate substance is selected from the group consisting of silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

In at least one embodiment, the formulation comprises a direct dye. Preferred among the direct dyes are the following compounds, alone or in combination with one another: Hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydroxyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof. Particularly preferred among the aforesaid direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydro-xyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)-amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof. In at least one embodiment, the total quantity of direct dyes in the formulation amounts to 0.01 to 15 wt.-%, preferably 0.1 to 10 wt.-%, most preferred 0.5 to 8 wt.-%.

In at least one embodiment, the formulation comprises a conditioning agent. In at least one embodiment, the conditioning agent is a water insoluble, water dispersible, non-volatile, liquid that forms emulsified, liquid particles. In at least one embodiment, the conditioning agent is a silicone (e.g., silicone oil, cationic silicone, silicone gum, high refractive silicone, or silicone resin), an organic conditioning oil (e.g., hydrocarbon oils, polyolefins, or fatty esters), a cationic conditioning surfactant, a high melting point fatty compound, or combinations thereof.

In at least one embodiment, the conditioning agent is a silicone, and the formulation comprises from 0.01% to 10%, or from 0.1% to 5% silicone conditioning agent, by total weight of the formulation. Suitable silicone conditioning agents, and optional suspending agents for the silicone, are described in U.S. Pat. No. 5,104,646. In at least one embodiment, the formulation comprises a silicone gum selected from the group consisting of polydimethylsiloxane, (polydimethylsiloxane) (methylvinylsiloxane) copolymer, poly(dimethylsiloxane) (diphenylsiloxane) (methylvinylsiloxane) copolymer, and mixtures thereof.

In at least one embodiment, the formulation comprises a terminal aminosilicone. “Terminal aminosilicone” as defined herein means silicone comprising one or more amino groups at one or both ends of the silicone backbone. In at least one embodiment, the formulation is substantially free of any silicone compound comprising pendant amino groups. In an embodiment, the formulation is substantially free of any silicone compound other than terminal aminosilicones. In at least one embodiment, the amino group at at least one terminus of the silicone backbone of the terminal aminosilicone is selected from the group consisting of primary amines, secondary amines and tertiary amines. In at least one embodiment, the formulation comprises from 0.1% to 20%, or from 0.5% to 10%, or from 1% to 6% terminal aminosilicone, by total weight of the formulation.

In at least one embodiment, the formulation comprises a cationic conditioning surfactant. In at least one embodiment, the formulation comprises from 0.05% to 3.0%, or from 0.075% to 2.0%, or from 0.1% to 1.0%, of cationic conditioning surfactant by total weight of the formulation. In at least one embodiment, the cationic conditioning surfactant is comprised in a lamellar gel matrix. In other words, the formulation comprises a lamellar gel matrix and the lamellar gel matrix comprises the cationic conditioning surfactant. In an embodiment, cationic conditioning surfactant is according to Formula (C):

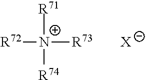

wherein at least one of R⁷¹, R⁷², R⁷³ and R⁷⁴ is selected from an aliphatic group of from 8 to 30 carbon atoms, an aromatic, alkoxy, polyoxyalkylene, alkylamido, hydroxyalkyl, aryl, or an alkylaryl group having up to 22 carbon atoms; the remainder of R⁷¹, R⁷², R⁷³ and R⁷⁴ are independently selected from the group consisting of an aliphatic group of from 1 to 22 carbon atoms, and an aromatic, alkoxy, polyoxyalkylene, alkylamido, hydroxyalkyl, aryl or alkylaryl group having up to 22 carbon atoms;
X is selected from the group consisting of: halogen, acetate, citrate, lactate, glycolate, phosphate, nitrate, sulfonate, sulfate, alkylsulfate, alkyl sulfonate, and combinations thereof.

In at least one embodiment, the cationic conditioning surfactant is selected from the group consisting of behenyl trimethyl ammonium chloride, methyl sulfate or ethyl sulfate, and stearyl trimethyl ammonium chloride, methyl sulfate or ethyl sulfate. It is believed that a longer alkyl group provides improved smoothness and soft feeling on wet and dry hair, compared to cationic surfactants with a shorter alkyl group. It is also believed that such cationic surfactants can provide reduced irritation, compared to those having a shorter alkyl group.

In at least one embodiment, the cationic surfactant is a di-long alkyl quaternized ammonium salt selected from the group consisting of: dialkyl (C14-C18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In at least one embodiment, the cationic surfactant is a tertiary amido amine having an alkyl group of from 12 to 22 carbons. The tertiary amido amine may be selected from the group consisting of stearamidopropyldimethyl-, stearamidopropyldiethyl-, stearamidoethyldiethyl-, stearamidoethyldimethyl-, palmitamidopropyldimethyl-, palmitamidopropyldiethyl-, palmitamidoethyldiethyl-, palmitamidoethyldimethyl-, behenamidopropyldimethyl-, behenamidopropyldiethyl-, behenamidoethyldiethyl-, behenamidoethyldimethyl-, arachidamidopropyldimethyl-, arachidamidopropyldiethyl-, arachidamidoethyldiethyl-, and arachidamidoethyldimethyl-amine, diethylaminoethylstearamide, and mixtures thereof. A tertiary amido amine may be used in combination with an acid. The acid is typically used as a salt-forming anion. In an embodiment, the acid is selected from the group consisting of lactic acid, malic acid, hydrochloric acid, 1-glumatic acid, acetic acid, citric acid, and mixtures thereof.

In at least one embodiment, the cationic surfactant is selected from the group consisting of cetyltrimethylammonium chloride (CTAC), stearyltrimethylammonium chloride (STAC), behentrimethylammonium methosulfate, stearoylamidopropyldimethyl amine (SAPDMA), distearyldimethylammonium chloride, and mixtures thereof.

In at least one embodiment, the formulation comprises a surfactant system. In at least one embodiment, the surfactant system comprises a surfactant selected from the group consisting of anionic surfactants, cationic surfactants, non-ionic surfactants, zwitterionic surfactants and/or amphoteric surfactants. In at least one embodiment, the formulation comprises a total amount of surfactant of from 0.01 wt.-% to 70 wt.-%, from 0.1 wt.-% to 40%, from 1 wt.-% to 30%, from 2 wt.-% to 20 wt. %.

In at least one embodiment, the formulation comprises an anionic surfactant. In at least one embodiment, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, alpha-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono, di-, and triethanolammonium, and analogous alkylammonium salts. In at least one embodiment, the anionic surfactant is the salt of an anionic surfactant comprising 12 to 14 carbon atoms. In at least one embodiment, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof.

In at least one embodiment, the formulation comprises an acylglycinate surfactant. In at least one embodiment, the acylglycinate surfactant conforms to the formula (Y):

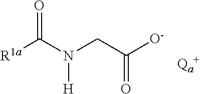

wherein
R^{1a} is a linear or branched, saturated alkyl group having 6 to 30, preferably 8 to 22, particularly preferably 8 to 18 carbon atoms or is a linear or branched, mono- or polyunsaturated alkenyl group having 6 to 30, preferably 8 to 22, particularly preferably 12 to 18 carbon atoms, and
Qₐ⁺ is a cation.

In at least one embodiment, Qₐ⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof.

In at least one embodiment, the acylglycinate surfactant is selected from sodium cocoylglycinate and potassium cocoylglycinate. In at least one embodiment, the acylglycinate surfactant is selected from those conforming to formula (Y), wherein R is C₁₂ alkyl or C₁₄ alkyl. In at least one embodiment, the acylglycinate surfactant is selected from those conforming to formula (Y), wherein R is C₁₆ alkyl or C₁₈ alkyl.

In at least one embodiment, the formulation comprises a glutamate surfactant corresponding to formula (Z) or a salt thereof:

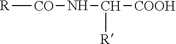

wherein
R′ is HOOC—CH2-CH2- or M⁺⁻OOC—CH2-CH2- wherein M⁺ is a cation;
R is a linear or branched, saturated alkyl group having 6 to 30, preferably 8 to 22, more preferably 8 to 18 carbon atoms or is a linear or branched, mono- or polyunsaturated alkenyl group having 6 to 30, preferably 8 to 22, more preferably 12 to 18 carbon atoms. In at least one embodiment, M⁺ is a metal cation. In at least one embodiment, M⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof.

In at least one embodiment, the glutamate surfactant is selected from sodium cocoyl glutamate and potassium cocoyl glutamate. In at least one embodiment, the glutamate surfactant is selected from those conforming to formula (Z), wherein R is C₁₂ alkyl or C₁₄ alkyl. In at least one embodiment, the glutamate surfactant is selected from those conforming to formula (Z), wherein R is C₁₆ alkyl or C₁₈ alkyl.

In at least one embodiment, the formulation comprises from 0.01 wt.-% to 30 wt.-%, preferably from 1 wt.-% to 25 wt.-%, more preferably from 5 wt.-% to 20 wt.-%, particularly preferably from 12 wt.-% to 18 wt.-% anionic surfactant.

In at least one embodiment, the amphoteric surfactants are selected from the group consisting of N—(C₁₂-C₁₈)-alkyl-beta-aminopropionates and N—(C₁₂-C₁₈)-alkyl-beta-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethyl-sulfopropylbetaine, amphosurfactants based on imidazoline (e.g. Miranol®, Steinapon®), preferably the sodium salt of 1-(beta-carboxymethyloxyethyl)-1-(carboxymethyl)-2-laurylimidazolinium; amine oxides, e.g. (C₁₂-C₁₈)-alkyldimethylamine oxides, fatty acid amidoalkyldimethylamine oxides, and mixtures thereof.

In at least one embodiment, the formulation comprises a betaine surfactant. Optionally, the betaine surfactant is selected from C₈- to C₁₈-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine, and combinations thereof. Optionally, the betaine surfactant is selected from C₈- to C₁₈-sulfobetaines. In at least one embodiment, the betaine surfactant is selected from the group consisting of cocodimethylsulfopropylbetaine, stearyldimethylsulfopropylbetaine, lauryldimethylsulfoethylbetaine, laurylbis(2-hydroxyethyl)sulfopropylbetaine, and combinations thereof. Optionally, the betaine surfactant is selected from carboxyl derivatives of imidazole, the C₈- to C₁₈-alkyldimethylammonium acetates, the C₈- to C₁₈-alkyldimethylcarbonylmethylammonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

In at least one embodiment, the formulation comprises from 0.5 wt.-% to 20 wt.-%, preferably from 1 wt.-% to 10 wt.-% amphoteric surfactant.

In at least one embodiment, the formulation comprises a surfactant system. In at least one embodiment, the surfactant system comprises at least one surfactant selected from the group consisting of lauryl sulfate, laureth sulfate, cocoamido-propylbetaine, sodium cocoylglutamate, lauroamphoacetate, and mixtures thereof. In at least one embodiment, the surfactant system comprises sodium laureth sulphate, sodium lauryl sulphate, and optionally cocamidopropyl betaine. In at least one embodiment, the surfactant system comprises sodium laureth sulphate, potassium cocoyl glutamate, and cocamidopropyl betaine.

In at least one embodiment, the formulation has a viscosity of from 0 cPs to 20,000 cPs. In at least one embodiment, the formulation has a viscosity of from 0.1 cPs to 10,000 cPs, or from 1 cPs to 5,000 cPs, or from 5 cPs to 3,500 cPs. Viscosity may be important for anti-drip reasons. Dripping can be inconvenient for the user. Furthermore, more viscous formulations can be useful for measured dispensing. In at least one embodiment, the formulation has a viscosity of from 0 cPs to 1,000 cPs. This viscosity range is advantageous when the formulation is in the form of a facial cleanser in view of the need for distribution on skin and ability to rinse off.

In at least one embodiment, the formulation further comprises a viscosity-modifying substance. The viscosity-modifying substance is preferably a thickening polymer.

In at least one embodiment, the thickening polymer is a polymer based on acrylamidomethylpropanesulfonic acid (AMPS®). These polymers, even at pH values of 7 or less, exhibit good thickening performance. Especially preferably, the thickening polymer is selected from the group consisting of homo- or copolymers of acrylamidomethylpropanesulfonic acid and salts thereof. Among the polymers just mentioned, preference is given to polymers having at least 20 mol-% of units based on acrylamidomethylpropanesulfonic acid and/or salts thereof, and particular preference to polymers having at least 50 mol-% of units based on acrylamidomethylpropanesulfonic acid and/or salts thereof, the mole figures relating in each case to the overall polymer. In the case of the copolymers, in addition to structural units based on acrylamidomethylpropanesulfonic acid and/or salts thereof, preferably one or more structural units based on the following comonomers are present in the copolymers: acrylic acid, methacrylic acid, acrylamide, dimethylacrylamide, vinylpyrrolidone (VP), hydroxyethyl acrylate, hydroxyethyl methacrylate, acrylic or methacrylic esters of ethoxylated alcohols RO—(CH₂CH₂O)ₘH, in which R is an alkyl radical having 12 to 30 carbon atoms and m is a number from 3 to 30, and CH₂═CH—COO—(CH₂CH₂—COO)ₙX, in which n is a number from 0 to 10 and X is a counterion and is preferably H⁺, Na⁺ and/or NH4⁺. The polymers selected from the group consisting of homo- or copolymers of acrylamidomethylpropanesulfonic acid and salts thereof may be crosslinked or non-crosslinked. In the case of crosslinking, they contain structural units based on monomers having 2 or more olefinic double bonds. In the case of crosslinking, preferably from 0.1 to 10 mol-% of such structural units are present in the homo- or copolymers, based on the overall polymer. If one or more structural units based on acrylamidomethylpropanesulfonic acid and/or salts thereof in the homo- or copolymers of acrylamidomethylpropanesulfonic acid and/or salts thereof have one or more counterions other than H+, these other counterions are preferably selected from the group consisting of Na+ and NH4+. Suitable polymers are mentioned in publications including EP-0816403, EP-1069142, EP-1116733 and DE-10 2009 014877 (Clariant), EP-1347736 (L'Oréal) or EP-1496081 (Seppic). Examples include: Aristoflex® AVC (Ammonium Acryloyldimethyltaurate/VP Copolymer), Aristoflex® AVS (Sodium Acryloyldimethyltaurate/VP Crosspolymer), Aristoflex® TAC (Ammonium Acryloyl Dimethyltaurate Carboxyethyl Acrylate Crosspolymer), Hostacerin® AMPS (Ammonium Polyacryloyldimethyl Taurate), Aristoflex® HMB (Ammonium Acryloyldimethyltaurate/Beheneth-25 Methacrylate Crosspolymer), Aristoflex® BLV (Ammonium Acryloyldimethyltaurate/Beheneth-25 Methacrylate Crosspolymer), Aristoflex® HMS (Ammonium Acryloyldimethyltaurate/Steareth-25 Methacrylate Crosspolymer), Aristoflex® SNC (Ammonium Acryloyldimethyltaurate/Steareth-8 Methacrylate Copolymer), Aristoflex® LNC (Ammonium Acryloyldimethyltaurate/Laureth-7 Methacrylate Copolymer) or Sepinov® EMT 10 (Hydroxyethyl Acrylate/Sodium Acryloyldimethyl Taurate Copolymer), Sepigel® 305.

In at least one embodiment, the thickening polymer is selected from the group consisting of: copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohols, and a third monomer type, which is chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes; hydroxypropyl starch phosphate; polyacrylamide; copolymers of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer); and mixtures thereof.

In at least one embodiment, the formulation has a pH value of from 2.0 to 12.0, preferably from 3.0 to 9.0, more preferably from 4.5 to 7.5. By varying the pH value, a formulation can be made available that is suitable for different applications.

In at least one embodiment, the formulation comprises an alkalizing agent or pH adjusting agent. In at least one embodiment, ammonia or caustic soda is suitable, but water-soluble, physiologically tolerable salts of organic or inorganic bases can also be considered. Optionally, the pH adjusting agent is selected from ammonium hydrogen carbonate, ammonia, monoethanolamine, ammonium carbonate. In at least one embodiment, the alkalizing agent or pH adjusting agent is selected from the group consisting of 2-amino-2-methyl-1-propanol, 2-amino-2-methyl-1,3-propanediol, 2-amino-2-ethyl-1,3-propanediol, tris(hydroxymethyl)-aminomethane, 2-amino-1-butanol, tris(2-hydroxypropyl)-amine, 2,2-iminobisethanol, lysine, iminourea (guanidine carbonate), tetrahydro-1,4-oxazine, 2-amino-5-guanidin-valeric acid, 2-aminoethansulfonic acid, diethanolamine, triethanolamine, N-methyl ethanolamine, isopropanolamine, diisopropanolamine, triisopropanolamine, glucamine, sodium hydroxide, potassium hydroxide, lithium hydroxide, magnesium oxide, and mixtures thereof.

To establish an acidic pH value, an acid can be included. In at least one embodiment, the formulation comprises an acid selected from the group consisting of hydrochloric acid, phosphoric acid, acetic acid, formic acid, sulfuric acid, citric acid, and mixtures thereof. Citric acid is most preferred in that it has high consumer acceptance. In at least one embodiment, the acidic pH is adjusted with a buffer such as a phosphate buffer, a TRIS buffer or a citric buffer. The buffers may be used alone or in combination with an acid.

The formulation of the present invention may be prepared by methods known in the art. For example, the formulation may be prepared by mixing its ingredients, preferably at ambient temperature (20° C.) and ambient pressure (1013 mbar).

The examples which follow are intended to illustrate the subject matter of the invention without restricting it thereto.

### EXAMPLES

### Materials and Methods

GlucoTain® GEM is commercially available from Clariant. Its chemical name is N-Sunflower-acyl-N-methyl-glucamin; its INCI name is Sunfloweroyl Methylglucamide. GlucoTain® GEM is prepared by reaction of N-Methyl Glucamine with high oleic sunflower oil in propylene glycol. The active content of GlucoTain® GEM is 72.5-75.5 wt.-%. GlucoTain® GEM contains 18-22 wt.-% of propylene glycol and max. 6.5 wt.-% of glycerol. The Renewable Carbon Index (RCI) of GlucoTain® GEM (without solvent) is 96% (determined according to DIN ISO 16128). The HLB value of GlucoTain® GEM is 7.1 (volumetric HLB value determined with Molecular Modeling Pro version 6.3.6).

Glucamide 68 RM is prepared by reaction of N-Methyl Glucamine with C16/C18 fatty acid methyl ester in propylene glycol. Glucamide 68 RM contains 80 wt.-% of C16/C18 methyl glucamide (compound of Formula (I) wherein Ra is a mixture of C15 and C17 alkyl and Rb is methyl) and 20 wt.-% of propylene glycol.

The viscosity was measured at 20° C. using a Brookfield viscometer DV-II+ Pro Extra at 20 rpm.

### Example 1

Emulsions/gel networks (10% active in water) were prepared using GlucoTain® GEM and cetearyl alcohol as follows:
I Mix GlucoTain® GEM and cetearyl alcohol in the main beaker and heat in a water bath to 80° C.
II Heat water in a water bath to 80° C.
III Add II to I and mix with a high shear mixer (Ultra-Turax) at 15.000 rpm for 2 min.
IV Cool down to 35° C. without stirring (waterbath at 35° C.).
V At 35° C. mix again with a high shear mixer (Ultra-Turax) at 15.000 rpm for 1 min. Cool down to RT without stirring.
VI After 24 h measure viscosity at 20° C.

| | GlucoTain ® | Cetearyl | | Viscosity of |
|---|---|---|---|---|
| | GEM | Alcohol | Water | the emulsion |
| Example | [wt.-%] | [wt.-%] | [wt.-%] | [mPas] |
| 1a | 10 | 0 | 90 | 2300 |
| 1b | 9 | 1 | 90 | 6250 |
| 1c | 8 | 2 | 90 | 5740 |
| 1d | 7 | 3 | 90 | 5230 |
| 1e | 6 | 4 | 90 | 8510 |
| 1f | 5 | 5 | 90 | 12850 |
| 1g | 4 | 6 | 90 | 13650 |
| 1h | 3 | 7 | 90 | 22400 |
| 1i | 2 | 8 | 90 | 25200 |
| 1j | 1 | 9 | 90 | 22000 |
| 1k | 0 | 10 | 90 | — |

All emulsions showed an increased viscosity. The highest viscosities were achieved when GlucoTain® GEM and cetearyl alcohol were used in ratios of 3:7, 2:8 and 1:9, respectively. Under an optical microscope (40× magnification) with polarized light, Maltese crosses were visible in these cases, indicating the formation of a gel network/liquid crystalline structure.

### Example 2

Flakes were prepared from GlucoTain® GEM and cetearyl alcohol in the following ratios:

| Example | 2a | 2b | 2c |
|---|---|---|---|
| Weight ratio of GlucoTain ® GEM | 2.5:7.5 | 2:8 | 1:9 |
| to Cetearyl Alcohol | | | |
| Optical appearance (viscosity) | Good | Good | Good |

The flakes were prepared as follows (laboratory process): GlucoTain® GEM and cetearyl alcohol were mixed with each other. The resulting mixture was heated to a temperature of about 80° C. The resulting melt was poured onto a cold surface. The resulting solidified product was broken into flakes.

The optical appearance (viscosity) was good in all cases.

### Example 3

Flakes were prepared from GlucoTain® GEM and Fatty Acid in the following ratios:

| Example | 3a | 3b | 3c | 3d | 3e |
|---|---|---|---|---|---|
| Weight ratio of GlucoTain ® GEM | 5:5 | 4:6 | 3:7 | 2:8 | 1:9 |
| to Fatty Acid | | | | | |
| Optical appearance (viscosity) | Good | Good | Good | Good | Good |

The flakes were prepared as follows (laboratory process): GlucoTain® GEM and Fatty Acid were mixed with each other. The resulting mixture was heated to a temperature of about 80° C. The resulting melt was poured onto a cold surface. The resulting solidified product was broken into flakes.

The optical appearance (viscosity) was good in all cases.

Composition of the Fatty Acid used in this experiment: C12: max. 1%; C14: max. 2%; C16: 54-62%; C18: 42-45%; C18-1: max. 1%; Others: max. 1%.

### Example 4

Mixtures of Plantasens® Emulsifier SFO (Sunflower Seed Oil Sorbitol Esters) and GlucoTain® GEM were studied with regard to their emulsifying properties. 10% of these mixtures were emulsified in water with an Ultra-Turrax, and the viscosity was measured after 24 h. The best results were achieved when Plantasens® Emulsifier SFO and GlucoTain® GEM were used in ratios of 7:3 and 6:4, respectively:

| | Ratio of Plantasens ® | Viscosity of | |
|---|---|---|---|
| | Emulsifier SFO to | the emulsion | Optical appearance |
| Example | GlucoTain ® GEM | [mPas] | of the emulsion |
| 4a | 7:3 | 605 | Homogeneous, |
| | | | white |
| 4b | 6:4 | 1115 | Homogeneous, |
| | | | white |

*Weight ratio

### Example 5

Emulsions (10% active in water) were prepared and the viscosity was determined:

| | | | | | Viscosity of |
|---|---|---|---|---|---|
| | Glucamide | Glyceryl | Cetearyl | | the |
| | 68 RM | Stearate | Alcohol | Water | emulsion |
| Example | [wt.-%] | [wt.-%] | [wt.-%] | [wt.-%] | [mPas] |
| 5a | 1 | 4.5 | 4.5 | 90 | 38400 |
| 5b | 2 | 4 | 4 | 90 | 38500 |
| 5c | 3 | 3.5 | 3.5 | 90 | 32500 |
| 5d | 4 | 3 | 3 | 90 | 20500 |

The highest viscosities were achieved when Glucamide 68 RM and the co-emulsifiers were used in ratios of 1:9, 2:8 and 3:7, respectively.

### Example 6

Emulsions (6-10% active in water, 10% oil phase) were prepared and the viscosity was determined:

| | Glucamide 68 | | | | |
|---|---|---|---|---|---|
| | RM/Glyceryl | Cetearyl | | | |
| | Stearate/ | Glucoside | | | |
| | Cetearyl | and Cetearyl | | | Viscosity |
| | Alcohol (2.5/ | Alcohol | Oil | | of the |
| Exam- | 3.75/3.75) | (Montanov 68) | phase* | Water | emulsion |
| ple | [wt.-%] | [wt.-%] | [wt.-%] | [wt.-%] | [mPas] |
| 6a | 6 | — | 10 | 84 | 13500 |
| 6b | — | 6 | 10 | 84 | 6900 |
| 6c | 8 | — | 10 | 82 | 21400 |
| 6d | — | 8 | 10 | 82 | 19500 |
| 6e | 10 | — | 10 | 80 | 32000 |
| 6f | — | 10 | 10 | 80 | 26300 |

*Oil phase = Dicaprylylether:Caprylic/Capric Triglyceride 1:2

Higher viscosities were achieved when an emulsifier system based on Glucamide 68 RM was used, as compared to an emulsifier system based on Cetearyl Glucoside, which can be considered a natural benchmark, but with a higher carbon footprint.

### Example 7

Emulsions (10% active in water) were prepared and the viscosity was determined:

| | Glucamide 68 | | | |
|---|---|---|---|---|
| | RM/Glyceryl | Cetearyl | | |
| | Stearate/ | Glucoside and | | |
| | Cetearyl | Cetearyl | | Viscosity |
| | Alcohol (2.5/ | Alcohol | | of the |
| | 3.75/3.75) | (Montanov 68) | Water | emulsion |
| Example | [wt.-%] | [wt.-%] | [wt.-%] | [mPas] |
| 7a | 10 | — | 90 | 28700 |
| 7b | — | 10 | 90 | 21500 |

A higher viscosity was achieved when an emulsifier system based on Glucamide 68 RM was used, as compared to an emulsifier system based on Cetearyl Glucoside, which can be considered a natural benchmark, but with a higher carbon footprint.

### Example 8

An emulsion (10% active in water) was prepared using GlucoTain® GEM and sorbitan stearate:

| GlucoTain ® GEM | Sorbitan Stearate | Water | Viscosity of the |
|---|---|---|---|
| [wt.-%] | [wt.-%] | [wt.-%] | emulsion [mPas] |
| 1 | 9 | 90 | 8900 |

The viscosity was good. Under an optical microscope with polarized light, Maltese crosses were visible, indicating the formation of a liquid crystalline structure.

### Example 9

An emulsion (5% active in water) was prepared using GlucoTaino GEM, cetearyl alcohol and sorbitan stearate:

| GlucoTain ® GEM/ | | | |
|---|---|---|---|
| Cetearyl Alcohol | Sorbitan Stearate | Water | Viscosity of the |
| (2:8) [wt.-%] | [wt.-%] | [wt.-%] | emulsion [mPas] |
| 6 | 4 | 190 | 10740 |

The viscosity was good. Under an optical microscope with polarized light, Maltese crosses were visible, indicating the formation of a liquid crystalline structure.

### Example 10

Formulation examples: If not stated otherwise, % as used herein refers to wt.-%.

Body lotion

| A | Water | ad 100% |
|---|---|---|
| | Glycerin 85% | 3.00% |
| B | GlucoTain ® GEM (Clariant) | 0.60% |
| | Sunfloweroyl Methylglucamide | |
| | Cetearyl Alcohol | 2.70% |
| | Glyceryl Stearate | 2.70% |
| | Plantasens ® Sweet Almond Oil Ref (Clariant) | 5.00% |
| | Prunus Amygdalus Dulcis (Sweet Almond) Oil | |
| | Plantasens ® Shea Butter Refined (Clariant) | 4.00% |
| | Butyrospermum Parkii (Shea Butter) | |
| | Plantasens ® Olive LD (Clariant) | 3.00% |
| | Hydrogenated Ethylhexyl Olivate (and) | |
| | Hydrogenated Olive Oil Unsaponifiables | |
| C | Nipaguard ® SCE (Clariant) | 1.00% |
| | Sorbitan Caprylate (and) Propanediol (and) | |
| | Benzoic Acid | |
| | Perfume | 0.20% |

Face care

| | | |
|---|---|---|
| A | Water | ad 100% |
| | Glycerin | 2.00% |
| | Aristoflex ® Silk (Clariant) | 1.00% |
| | Sodium Polyacryloyldimethyl Taurate | |
| B | Plantasens ® Carefeel Light (Clariant) | 4.00% |
| | Ethylhexyl Olivate | |
| | Plantasens ® Olive LD (Clariant) | 2.00% |
| | Hydrogenated Ethylhexyl Olivate (and) | |
| | Hydrogenated Olea Europea | |
| | (Olive) Oil Unsaponifiable | |
| | Plantasens ® Squalane (Clariant) | 4.00% |
| | Squalane | |
| | GlucoTain ® GEM (Clariant) | 0.90% |
| | Sunfloweroyl Methylglucamide | |
| | Cetearyl Alcohol | 2.10% |
| | Rainforest Cupuaçu Butter (Beraca) | 0.50% |
| | Theobroma Grandiflorum Seed Butter (and) | |
| | Tocopherol | |
| C | Water | 5.00% |
| | Trisodium Citrate | 0.10% |
| D | RedSnow ® (Clariant) | 1.00% |
| | Propanediol (and) Water (and) Camellia | |
| | Japonica Flower Extract | |
| | LotuStem ® (Clariant) | 1.00% |
| | Butylene Glycol (and) Water (and) Andrographis | |
| | Paniculata Extract | |
| | Nipaguard ® SCP (Clariant) | 1.00% |
| | Sorbitan Caprylate (and) Phenoxyethanol | |
| | Perfume | 0.20% |
| E | Citric Acid | q.s to pH 5.5 |

Beauty balm

| | | |
|---|---|---|
| A | Water | ad 100% |
| | Aristoflex ® Velvet (Clariant) | 0.50% |
| | Polyacrylate Crosspolymer-11 | |
| | Glycerin (85%) | 3.00% |
| B | GlucoTain ® GEM (Clariant) | 0.50% |
| | Sunfloweroyl Methylglucamide | |
| | Cetearyl Alcohol | 4.00% |
| | Aristoflex ® AVC (Clariant) | 1.00% |
| | Ammonium Acryloyldimethyltaurate/VP Copolymer | |
| | Plantasens ® Abyssinian Oil (Clariant) | 3.00% |
| | Crambe Abyssinica Seed Oil | |
| | Octyldodecanol | 5.00% |
| | Isodecyl Neopentanoate | 3.00% |
| C | Plantasens ® Natural Vitamin E (Clariant) | 0.50% |
| | Tocopherol | |
| | Gatuline Age Defense 2 | 1.00% |
| | Water (and) Juglans Regia (Walnut) Seed Extract | |
| | Nipaguard ® SCP (Clariant) | 1.35% |
| | Phenoxyethanol (and) Sorbitan Caprylate | |
| | Perfume Ulysse (SOZIO) | 0.30% |
| D | Citric Acid solution | q.s. pH 5.50 |

Self-tanning cream SPF 10 (calculated)

| | | |
|---|---|---|
| A | Water | ad 100% |
| | Glycerin | 3.00% |
| | Aristoflex ® Silk (Clariant) | 1.00% |
| | Sodium Polyacryloyldimethyl Taurate | |
| B | GlucoTain ® GEM (Clariant) | 1.40% |
| | Sunfloweroyl Methylglucamide | |
| | Cetearyl Alcohol | 4.10% |
| | Xiameter ® DC 200 | 2.00% |
| | Dimethicone | |
| | C12-15 Alkyl Benzoate | 2.00% |
| | Eclipsogen ® AVB (Clariant) | 2.00% |
| | Butyl Methoxybenzoylmethane | |
| | Eclipsogen ® HOS (Clariant) | 3.00% |
| | Homosalate | |
| | Eclipsogen ® OC (Clariant) | 5.00% |
| | Octocrylene | |
| C | Water | 10.00% |
| | Dihydroxyacetone | 5.00% |
| D | Phenonip ® ME (Clariant) | 1.00% |
| | Phenoxyethanol (and) Methylparaben (and) | |
| | Ethylparaben | |
| E | Perfume | 0.30% |
| | Citric Acid | q.s. to pH 5.5 |

Jelluminator

| A | Water | Ad 100% |
|---|---|---|
| | Glycerin (85%) | 10.00% |
| | Aristoflex ® BLV (Clariant) | 0.65% |
| | Ammonium Acryloyldimethyltaurate/Beheneth-25 | |
| | Methacrylate Crosspolymer | |
| | Aristoflex ® HMB (Clariant) | 0.10% |
| | Ammonium Acryloyldimethyltaurate/Beheneth-25 | |
| | Methacrylate Crosspolymer | |
| B | GlucoTain ® GEM (Clariant) | 0.50% |
| | Sunfloweroyl Methylglucamide | |
| | Cetearyl Alcohol | 4.50% |
| | Plantasens ® Carefeel Light (Clariant) | 2.00% |
| | Ethylhexyl Olivate | |
| | Plantasens ® Olive LD (Clariant) | 1.00% |
| | Hydrogenated Ethylhexyl Olivate (and) Hydrogenated | |
| | Olive Oil Unsaponifiables | |
| C | MearlMica ® CF | 6.50% |
| | Mica | |
| | Gemtone ® Tan Opal | 3.00% |
| | Mica (and) Titanium Dioxide (and) Iron Oxides | |
| | Timiron ® SynBeam Gold | 3.00% |
| | Synthetic Fluorphlogopite (and) CI 77891 (and) | |
| | Tin Oxide | |
| | 1,2-Propanediol | 20.00% |
| D | Nipaguard ® SCP (Clariant) | 1.00% |
| | Phenoxyethanol (and) Sorbitan Caprylate | |
| | Eosidin ™ (Clariant) | 1.00% |
| | Propanediol (and) Water (and) Citrus Unshiu | |
| | Peel Extract | |
| | Fragrance | 0.20% |

Makeup remover

| A | Water | Ad 100% |
|---|---|---|
| | Xanthan Gum | 0.50% |
| | Glycerin (85%) | 3.00% |
| B | GlucoTain ® GEM (Clariant) | 2.15% |
| | Sunfloweroyl Methylglucamide | |
| | Plantasens ® Refined Olive Oil (Clariant) | 5.00% |
| | Olea Europaea (Olive) Fruit Oil | |
| | Plantasens ® Refined Sweet Almond Oil (Clariant) | 1.00% |
| | Prunus Amygladus Dulcis (Sweet Almond) Oil | |
| | Cetearyl Alcohol | 2.50% |
| | Glyceryl Stearate | 2.50% |
| C | Nipaguard ® SCE (Clariant) | 1.00% |
| | Sorbitan caprylate, Propanediol, Benzoic Acid | |
| | Benzoic Acid | 0.30% |
| | Fragrance | 0.20% |

1.-15. (canceled)

16. A composition comprising
(A) at least one compound of Formula (I), 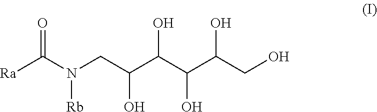
wherein
Ra is selected from saturated or unsaturated hydrocarbon chains having 5 to 23 carbon atoms, and mixtures thereof; or Ra—C═O is derived from high oleic sunflower oil, sunflower oil, olive oil, soybean oil, rapeseed oil or palm oil;
Rb is selected from C₁-C₄ alkyl;
(B) at least one co-emulsifier.

17. The composition according to claim 16, wherein
Ra is selected from saturated or unsaturated hydrocarbon chains having 11 to 21 carbon atoms, and mixtures thereof; or Ra—C═O is derived from high oleic sunflower oil, sunflower oil, olive oil, soybean oil or rapeseed oil; and
Rb is selected from methyl and ethyl.

18. The composition according to claim 16, wherein the at least one co-emulsifier (component B) is selected from fatty alcohols having 12 to 22 carbon atoms, fatty acids having 12 to 22 carbon atoms, esters of glycerol based on one or more C12-C22 fatty acids, esters of sorbitan based on one or more C8-C22 fatty acids, esters of sorbitol based on one or more C12-C22 fatty acids, esters of polyglycerol based on one or more C8-C22 fatty acids, esters of sucrose based on one or more C12-C22 fatty acids, esters of phosphoric acid based on one or more C12-C22 fatty alcohols or ethoxylated C12-C22 fatty alcohols, C12-C22 fatty alcohol ethoxylates, esters of lactic acid based on a C12-C22 fatty acid, and mixtures thereof.

19. The combination according to claim 16, wherein the at least one co-emulsifier (component B) is selected from cetearyl alcohol, cetyl alcohol, stearyl alcohol, stearic acid, palmitic acid, glyceryl stearate, sorbitan stearate, sorbitol esters derived from sunflower oil or high oleic sunflower oil, and mixtures thereof.

20. The composition according to claim 16, wherein the at least one co-emulsifier (component B) is selected from fatty alcohols, fatty acids, glyceryl esters, sorbitan esters, sorbitol esters, polyglyceryl esters, sucrose esters, phosphates, fatty alcohol ethoxylates, lactylates, and mixtures thereof.

21. The composition according to claim 16, wherein a weight ratio of (A) the at least one compound of Formula (I) to (B) the at least one co-emulsifier is from 5:1 to 1:30.

22. The composition according to claim 16, wherein a weight ratio of (A) the at least one compound of Formula (I) to (B) the at least one co-emulsifier is from 2:1 to 1:20.

23. The composition according to claim 16, wherein
Ra—C═O is derived from high oleic sunflower oil, sunflower oil, olive oil, soybean oil or rapeseed oil;
the composition comprises one or more fatty alcohols, one or more fatty acids having 12 to 22 carbon atoms, one or more sorbitol esters, or mixtures of these; and
a weight ratio of (A) the at least one compound of Formula (I) to (B) the at least one co-emulsifier from 3:7 to 1:9.

24. The composition according to claim 16, wherein
the composition comprises
a mixture of compounds according to Formula (I), wherein Ra is a saturated hydrocarbon chain having 15 or 17 carbon atoms and Rb is ethyl or methyl; and
one or more glyceryl esters, one or more sorbitan esters, one or more fatty alcohols, or mixtures of these, and
a weight ratio of (A) the at least one compound of Formula (I) to (B) the at least one co-emulsifier is from 3:7 to 1:9.

25. The composition according to claim 16, further comprising at least one alcohol selected from ethanol, propanol, isopropanol, propylene glycol, 1,3-propanediol, butanol, isobutanol, butylene glycol, dipropylene glycol, glycerol, and mixtures thereof.

26. The composition according to claim 25, wherein the composition comprises
(A) from 50 to 100 wt.-% of the at least one compound of Formula (I), based on the total weight of the composition;
(C) from 0 to 50 wt.-%, of the at least one alcohol, based on the total weight of the composition; and
(D) from 0 to 10 wt.-%, of water, based on the total weight of the composition.

27. The composition according to claim 25, wherein the composition comprises
(A) from 30 to 80 wt.-%, of the at least one compound of Formula (I), based on the total weight of the composition;
(C) from 0 to 40 wt.-%, of the at least one alcohol, based on the total weight of the composition; and
(D) from 0 to 60 wt.-%, of water, based on the total weight of the composition.

28. A composition comprising
(A) from 2 to 50 wt.-%, based on the total weight of the composition, the at least one compound of Formula (I) according to claim 16;
(B) from 40 to 95 wt.-%, based on the total weight of the composition, of at least one co-emulsifier;
(C) from 0 to 20 wt.-%, based on the total weight of the composition, of at least one alcohol;
(D) from 0 to 5 wt.-%, based on the total weight of the composition, of water.

29. The composition according to claim 28, wherein the at least one alcohol (component C) is selected from ethanol, propanol, isopropanol, propylene glycol, 1,3-propanediol, butanol, isobutanol, butylene glycol, dipropylene glycol, glycerol, and mixtures thereof.

30. A composition comprising
(A) from 0.5 to 5 wt.-%, based on the total weight of the composition, the at least one compound of Formula (I) according to claim 16,
(B) from 2 to 20 wt.-%, based on the total weight of the composition, of at least one co-emulsifier.

31. The composition according to claim 30, wherein the composition is a cosmetic formulation.
